(19) United States
(12) Patent Application Publication
(10) Pub. No.: US 2024/0366483 A1
(43) Pub. Date: Nov. 7, 2024

(54) TOPICAL ANTIMICROBIAL FORMULATION

(71) Applicant: EVERGREEN LAND LIMITED, Hong Kong (CN)

(72) Inventor: Nicolas Vidal, Hong Kong (CN)

(21) Appl. No.: 18/573,431

(22) PCT Filed: Jun. 21, 2022
(86) PCT No.: PCT/IB2022/055735

§ 371 Date: Dec. 21, 2023

(30) Foreign Application Priority Data
Jun. 21, 2021 (FR) FR2106594

(51) Int. Cl. A61K 8/365; A61K 8/36; A61K 8/37; A61K 8/41; A61K 8/73; A61K 8/92; A61Q 17/00

(52) U.S. Cl. CPC A61K 8/365; A61K 8/361; A61K 8/375; A61K 8/416; A61K 8/73; A61K 8/731; A61K 8/738; A61K 8/922; A61Q 17/005

(57) ABSTRACT

The invention relates to a topical antimicrobial formulation comprising an acidic substance comprising at least one organic acid diluted in an aqueous solvent while having an action deleterious against microbes, said acidic substance having an acidity constant pKa that is less than 6, as well as: between 1% and 10% by weight of acidic substance in the INCI base; a basic substance the quantity of which in the formulation is such that the pH is comprised between the pKa of the acidic substance and 8; between 0.1% and 15% by weight of a booster substance capable of biochemically interacting with the membrane of the microbes to facilitate the deleterious action of the acidic substance, said booster substance comprising at least one compound in the INCI base selected from amphiphilic compounds, chelating compounds having an ability to associate with divalent ions or sterols, or polyphenolic compounds.

The invention relates to a topical antimicrobial formulation.

It applies in particular to a formulation intended to be applied to the skin and in particular to the hands, for hygiene purposes and/or for avoiding the propagation of illnesses. Such formulations are generally used without rinsing, in addition to and/or in replacement for washing with water and soap, and can more particularly be used by care personnel in the context of health care or during travel, both personal and professional, as well as by children, in particular in a school environment, and more generally in the case of risk of exposure of the skin to a possibly contaminated surface.

The purpose of these formulations is to kill, deactivate or slow the growth of microbes harmful to human health, such as bacteria, yeasts, fungi and/or viruses.

Antimicrobial formulations are known, referred to as hydroalcoholic solutions, which contain an alcohol, in particular ethanol and/or isopropanol, mixed with an antimicrobial agent, for example a positively charged surfactant, of the quaternary ammonium type.

Antimicrobial formulations are also known that comprise an acidic substance comprising at least one organic acid diluted in an aqueous solvent while having a deleterious action against microbes, said acidic substance having an acidity constant pKa that is less than 6. These formulations prove to be particularly effective since, in addition to be easily biosourceable and biodegradable, they have low volatility in air and weak dermal absorption, which increases the efficacy of their antimicrobial action when applied to the skin of a user.

However, the known formulations are not entirely satisfactory, because of their low dermocompatibility caused by their acid pH, in particular less than the pKa of the acid, which is necessary for their efficacy. In particular, in the case of repeated applications, they may cause the appearance of undesirable effects for the skin, in particular drying, redness and/or irritations on the epidermis.

The invention aims to improve the prior art by proposing in particular a formulation that comprises an antimicrobial organic acidic substance, in order to benefit from the advantages relating to biosourceability, biodegradability and persistence on the epidermis, while having improved dermocompatibility.

For this purpose, the invention proposes a topical antimicrobial formulation comprising an acidic substance comprising at least one organic acid diluted in an aqueous solvent while having a deleterious action against microbes, said acidic substance having an acidity constant pKa that is less than 6, said formulation furthermore comprising:
between 1% and 10% by weight of acidic substance wherein the at least one organic acid used is referenced in the INCI base;
a basic substance comprising at least one base, the quantity of basic substance in the formulation being such that the pH of said formulation is comprised between the pKa of the acidic substance and 8;
between 0.1% and 15% by weight of a booster substance that is capable of biochemically interacting with the membrane of the microbes to facilitate the deleterious action of the acidic substance on said microbes, said booster substance comprising at least one compound referenced in the INCI base selected from amphiphilic compounds, chelating compounds having an ability to associate with divalent ions or sterols, and polyphenolic compounds.

Other particularities and advantages of the invention will emerge in the following description of various particular embodiments.

The invention relates to a topical antimicrobial formulation, in particular intended to be applied to the skin and in particular to the hands of a user for hygiene purposes and/or for combating the propagation of illnesses.

The formulation comprises an acidic substance comprising at least one organic acid diluted in an aqueous solvent while having a deleterious action against microbes. For optimum antimicrobial efficacy, the acidic substance has an acidity constant pKa that is less than 6. In the case of a polyacid, the pKa in question is that of the weakest acid.

The acidic substance may in particular have a virucidal activity, for example against viruses such as:
herpes, in particular EHV1 (standing for “Equine Herpesvirus1”);
adenoviruses, in particular AdV5 (standing for “Adenovirus 5”);
coronaviruses, in particular SARS-CoV-2 (standing for “Severe Acute Respiratory Syndrome Coronavirus 2”).

The acidic substance may also have other antimicrobial functions, in particular against bacteria (antibacterial), yeasts (yeasticidal) and/or fungi (antifungal).

The organic acid is in particular selected from carboxylic acids, i.e. acids having a carboxyl group. Among these acids, the following components are in particular found: lactic acid, citric acid, tartaric acid, acetic acid, adipic acid, anisic acid, malic acid, succinic acid, benzoic acid, cholic acid, ascorbic acid, dehydroacetic acid, deoxycholic acid, glucoronic acid, glycolic acid, fumaric acid, formic acid, gluconic acid, glutamic acid, guanylic acid, folic acid, fulvic acid, sorbic acid, glycyrrhizic acid, kojic acid, itaconic acid, mellisic acid, hyaluronic acid, amino acids, glutaric acid, glyoxylic acid, humic acids, hippuric acid, inosic acid, levulinic acid, perillic acid, quinic acid, tauric acid, ribonic acid, salicylic acid, shikimic acid, trenaxamic acid, thujic acid, tiglic acid, ursolic acid, usnic acid, vanillic acid, valproic acid.

The organic acid may in particular be selected from the acids coming from plant oils, such as: safflower acids, colza acids, coconut and copra acids, jojoba acids, palm acids, maize acids, soya acids, uric acid, as well as fatty acids, in particular fatty acids comprising between 3 and 24 carbon atoms (C3 to C24), including unsaturated and functionalised fatty acids.

To provide its dermatological compatibility, the formulation comprises between 1% and 10% by weight, and preferably between 1% and 5% by weight, of acidic substance in which the at least one organic acid used is referenced in the INCI base (standing for “International Nomenclature of Cosmetic Ingredients”).

In particular, the acidic substance comprises an organic acid or a mixture of organic acids, the individual concentrations of which are arranged to confer on the formulation a margin of safety greater than 100 (MOS, standing for “margin of safety”). This MOS margin is calculated in accordance with standard methods in the field of toxicology, from the dermal absorption and from the NOAEL value (standing for “No Observable Adverse Effect Level”) of each component of the formulation, as well as from the exposure scenario.

In advantageous examples, the acidic substance comprises at least one organic acid selected from lactic acid, lauric acid, citric acid, oleic acid or a mixture of at least two of these compounds.

The formulation furthermore comprises a basic substance comprising at least one base, the quantity of basic substance in said formulation being such that the pH of said formulation is comprised between the pKa of the acidic substance and 8.

Adding a basic substance makes it possible to limit the total acidity of the formulation, in order to improve the dermocompatibility thereof, and thus to limit the risk of irritations and/or redness, in particular after several applications to the skin of a user. In particular, the formulation may comprise a quantity of basic substance adapted for conferring on it a pH of around 5, which corresponds approximately to the minimum pH value of a human skin in good health.

The basic substance comprises at least one base selected from organic acid salts, in particular sodium lactate and/or potassium lactate, clays, in particular bentonite and/or montmorillonite, sodium hydroxide, potassium hydroxide, triethanolamine, butylethanolamine, disodium carbonates, dipotassium carbonates, disodium phosphates, dipotassium phosphates, or a mixture of at least two of these compounds.

Advantageously, the basic substance comprises sodium hydroxide, optionally mixed with a clay, in particular based on bentonite and/or montmorillonite.

When they are mixed in a formulation with a pH higher than their respective pKa constants, organic acids react chemically to produce salts, for example sodium salt or potassium salt, according to the acid or acids used, which greatly attenuates or even deactivates their antimicrobial properties, and thus reduces the efficacy of the formulation.

In particular, for each organic acid present in the formulation, the concentration of its organic form decreases even more as the pH value of said formulation increases. Thus, for a pH value lying between the pKa of a given organic acid and a pKa +1 value, the concentration of its acid form is 5% greater than the total concentration of said organic acid, whereas this percentage drops to 0.5% for a pH lying between pKa +1 and pKa +2.

To overcome these drawbacks, the formulation furthermore comprises between 0.1% and 15% by weight, and preferably between 1% and 10% by weight, of a booster substance that is capable of biochemically interacting with the membrane of the microbes to facilitate the deleterious action of the acidic substance on said microbes. In particular, the booster substance makes it possible to stimulate the deleterious action of the acidic substance.

The booster substance comprises at least one compound referenced in the INCI base selected from:
amphiphilic compounds;
chelating compounds having an ability to associate with divalent ions and sterols;
polyphenolic compounds.

Thus, adding a booster substance to the formulation makes it possible to benefit from the antimicrobial action of the acidic substance, and this despite a lower concentration of acidic forms due to the presence of the basic substance. In this way a formulation is obtained that has both good antimicrobial properties and good compatibility with the human skin.

The booster substance may comprise at least one amphiphilic compound having a critical micelle concentration (CMC) value of between 1 μM and 400 mM, in particular between 10 μM and 100 mM.

In particular, the booster substance comprises at least one amphiphilic compound selected from glyceryl laurate, glyceryl caprate, glyceryl caprylate, glyceryl palmitate, glyceryl stearate, quaternary ammoniums, saponins or a mixture of at least two of these compounds. The amphiphilic compound may also be selected in the INCI base from fatty alcohols, ethoxylated alcohols, an amphiphilic compound with betaine, phosphate or sulfate group, alkylated sugars, polyglyceryl, polysorbate, or a mixture of at least two of these compounds.

Advantageously, the formulation may in particular comprise:
between 5% and 7% by weight of glyceryl stearate;
between 0.5% and 3.5% by weight of glyceryl laurate;
between 0.2% and 0.7% by weight of xanthan.

Amphiphilic compounds have a molecular structure with a hydrophobic part having an affinity for membrane lipids and a hydrophilic part having an affinity for aqueous environments. They can interact with the membrane lipidic bilayer of the microbes, in order to destabilise the membrane thereof. Indeed, since this lipidic bilayer is mainly composed of phospholipids, some amphiphilic compounds can reduce the coherence thereof, and thus facilitate penetration with the membrane of the antimicrobial acidic substance.

The booster substance may also comprise at least one chelating compound that has a LogK constant of interaction with divalent ions or sterols greater than 4, and in particular greater than 8. This chelating compound can be selected from EDTA (ethylenediaminetetraacetic acid) or one of the salts thereof, cyclodextrin or one of the derivatives thereof, phytic acid or one of the salts thereof, oxalic acid or one of the salts thereof, or a mixture of at least two of these compounds.

Indeed, some microbe membranes comprise divalent ions, such as magnesium and calcium, or sterols, which increase the stability thereof. Thus, the use of chelating compounds with suitable LogK constants makes it possible to destroy the effect of the divalent ions and/or of the sterols and to reduce the stability of the membrane, which facilitates the penetration of said membrane by the antimicrobial acidic substance.

The booster substance may also comprise at least one polyphenolic compound, in particular selected from quercetin, oleuropein, cafeic acid, gallic acid, epigallocatechin, epigallocatechin-gallate, tannins, tannic acids, fulvic acids, humic acids, plant extracts, in particular based on olive, tea, cocoa bean, coffee or cassia bark, or a mixture of at least two of these compounds.

In a known manner, the presence of phenolic and/or polyphenolic compounds can contribute to the destabilisation of the microbe membranes, by interaction between the hydroxyl/benzoic groups of said compounds and the proteins of said membranes.

As with the acidic substance, the booster substance comprises compounds the individual concentrations of which are arranged to confer on the formulation a MOS margin greater than 100 MOS, in order to guarantee for said formulation good compatibility with the epidermis even in the case of repeated applications.

Advantageously, the booster substance comprises a mixture based on glyceryl stearate, glyceryl laurate and xanthan, optionally mixed with benzalkonium chloride, cetrimonium chloride and/or methyl cyclodextrin.

The formulation may furthermore comprise at least one additional compound having a dermocompatible function, and in particular a dermocosmetic function, in order to provide an additional beneficial effect for maintaining the epidermis.

In particular, the formulation may comprise at least one additional compound that has at least one dermocosmetic function selected from:
hydrating and/or moisturising functions, in order to increase the water content of the skin, in particular of the top layers (for the moisturising functions); and/or
emollient functions, in order to lubricate the surface of the skin to improve the appearance and feel thereof; and/or
relipidising functions, in order to reconstitute the lipids of the top layers of the skin.

For this purpose, the formulation may comprise at least one dermocosmetic compound selected from an oil, shea butter, glycerol, triheptanoin, p-menthanediol, dibutyl adipate, a fatty alcohol or a mixture of at least two of these compounds.

Advantageously, the formulation may in particular comprise:
between 1% and 5% by weight of glycerol;
between 1% and 6% by weight of almond oil.

The formulation may also comprise an additional compound having a homogenising function, in particular based on microcrystalline cellulose, in order to improve the distribution, in the aqueous solvent, of the various compounds of the formulation, in particular the compounds that have lipophilic groups.

The solvent of the formulation consists for the major part of water, the weight percentage of which is adjusted according to the weight percentage required for the other compounds of said formulation. Such a solvent has advantages in terms of dermocompatibility, in that it makes it possible to reduce the risks of allergies and/or irritation for the skin, in particular in the context of repeated applications.

The formulation may also comprise an additional organic solvent, in particular based on hexanediol.

Such additional compounds are referenced in the INCI base, and are also selected for satisfying the toxicological criterion relating to the MOS margin described previously.

### EXPERIMENTAL EXAMPLES

For each example of a test formulation presented below, the weight percentages relate to the total weight of said formulation.

### Example 1

A first series of tests were implemented in the laboratory to evaluate the efficacy of two different formulations against the EHV1 virus (Equine Herpes virus 1).

To do this, a first “test” formulation was prepared, called CMV42, which contains the following ingredients:
as antimicrobial organic acid:
   2% by weight of lactic acid;
as booster compounds:
   6.5% by weight of glyceryl stearate (amphiphilic);
   3% by weight of glyceryl laurate (amphiphilic);
   0.4% by weight of xanthan (amphiphilic).

A second “reference” formulation was also prepared, called CMV40, which is differentiated from the CMV42 formulation in that it does not contain lactic acid nor glyceryl laurate, and otherwise comprised the other compounds listed above in the same percentages.

These two formulations furthermore contain the following compounds, in the same percentages:
as base:
   0.05% by weight of a clay;
   sodium hydroxide, in a sufficient proportion for conferring on the formulation a pH of 5;
as dermocosmetic compounds:
   2% by weight of almond oil (hydrating);
   0.1% by weight of shea butter (hydrating);
   2% by weight of glycerol (moisturising);
0.05% by weight of microcrystalline cellulose;
1.4% by weight of hexanediol-based solvent;
the remainder in water.

These two formulations were each mixed, at a percentage of 97% and in accordance with four different dilution rates (10⁻³, 10⁻⁴, 10⁻⁵, 10⁻⁶), in a solution comprising cells coming from a rabbit renal cell line (RK13) infected by an EHV1 virus carrying a green fluorescent protein (GFP), with a viral load of 5.5×10⁸ ffu/ml. For each test sample, the time of exposure of the formulation to the virus was 5 minutes.

Next, a well was formed for each sample obtained on an examination slide, in order to be able to observe under the microscope the number of cells infected per well after this time of 5 minutes, compared with control wells containing only infected cells, without antimicrobial formulation.

For the “reference” formulation CMV40, very small reductions in the viral load were observed, ranging from 0.3 log (for a dilution at 10⁻⁴) to 0.56 log only (for a dilution at 10⁻³). In particular, the most diluted sample (10⁻⁶) had a negligible reduction in its viral load.

On the other hand, for the “test” formulation CMV42 according to the invention, much greater reductions in the viral load were observed, ranging from 1.56 log (for a dilution at 10⁻⁶) to 4.25 (for a dilution at 10⁻³). Thus this formulation has virucidal efficacy greater than 99.99% against the EHV1 virus, and proves to be effective even at high dilution levels.

This first test series allowed to highlight the efficacy of a formulation according to the invention comprising an organic acid, in particular based on lactic acid, compared with a similar formulation not containing organic acid.

### Example 2

Another “reference” formulation CMVTA was prepared containing only the following ingredients:
2% by weight of lactic acid as antimicrobial organic acid;
a basic substance based on sodium hydroxide, at a proportion adapted for conferring on this formulation a pH of 5;
the remainder in water.

This formulation was next tested in contact with a solution of cells infected by the EHV1 virus identical to that prepared for example 1, and in accordance with an experimentation protocol similar to this example 1, with the exception of the following parameters:
the contact time of the formulation with the virus was only 60 seconds; and
a single dilution level was implemented (10⁻³).

Because of the very small reduction observed for the viral load (0.28 log only), it was concluded that a formulation containing only the organic acid did not exhibit any efficacy against the EHV1 virus.

Thus the test series implemented in examples 1 and 2 presented above highlighted the efficacy against the EHV1 virus of a formulation according to the invention comprising both an organic acid and a booster substance, in particular in the following proportions:
2% by weight of lactic acid;
6.5% by weight of glyceryl stearate;
3% by weight of glyceryl laurate;
0.4% by weight of xanthan.

### Example 3.1

For this series of tests, a solution of cells infected by the EHV1 virus similar to that used in the previous examples 1 and 2 was prepared.

A formulation CMV42 was also prepared as described in example 1, as well as three other “test” formulations CMV41, CMV43 and CMV44 according to the invention, which differ from the CMV42 formulation by the addition of supplementary ingredients, namely:
for the CMV41 formulation:
   2% by weight of lauric acid as antimicrobial organic acid;
   0.5% by weight of methyl cyclodextrin as chelating booster compound;
for the CMV43 formulation:
   2% by weight of lauric acid as antimicrobial organic acid;
for the CMV44 formulation:
   0.5% by weight of methyl cyclodextrin as chelating booster compound.

Next each of these formulations was tested in accordance with a protocol similar to that of examples 1 and 2, with a single dilution rate (10⁻³) and a contact time of 30 seconds between each formulation and the virus.

Observing the samples under the microscope revealed a reduction in the viral load greater than 99% for each of the formulations tested, and more particularly:
for the CMV43 formulation, containing both lactic acid and lauric acid, but without the addition of methyl cyclodextrin: a reduction in the viral load of the order of 2.14 log; and
for the other formulations CMV41, CMV42 and CMV44, a reduction in the viral load of the order of 4.08 log, i.e. almost twice as great as the reduction in the viral load obtained with the CMV43 formulation.

This series of tests allowed to highlight an increased and very rapid virucidal efficacy against the EHV1 virus (as from 30 seconds of contact), for the formulations comprising the combination of following compounds:
6.5% by weight of glyceryl stearate;
3% by weight of glyceryl laurate;
0.4% by weight of xanthan; and
2% by weight of lactic acid, in particular:
   alone (formulations CMV42, CMV44); or
   in combination with (formulation CMV43):
      2% by weight of lauric acid; and
      0.5% by weight of methyl cyclodextrin as chelating booster compound.

### Example 3.2

Another “test” formulation CMV910 according to the invention was prepared, which comprises:
as antimicrobial organic acids:
   1% by weight of lactic acid;
   1% by weight of citric acid;
as booster compounds:
   6% by weight of glyceryl stearate (amphiphilic);
   1% by weight of glyceryl laurate (amphiphilic);
   0.5% by weight of xanthan (amphiphilic);
   0.13% by weight of benzalkonium chloride (amphiphilic);
as bases:
   sodium hydroxide, at a sufficient level to confer on the formulation a pH of 5;
as dermocosmetic compounds:
   7% by weight of almond oil (hydrating);
   2.5% by weight of glycerol (moisturising);
   1% by weight of triheptanoin (moisturising);
   2% by weight of dibutyl adipate (emollient);
   1.5% by weight of shea butter (emollient);
   5% by weight of a fatty alcohol (emollient);
the remainder in water.

This formulation was next tested in contact with a solution of cells infected by the EHV1 virus identical to those prepared for the previous examples, and in accordance with a similar experimentation protocol, with the exception of a contact time with the virus of 60 seconds and of a single dilution level of 10⁻³.

Observation of the tested sample under the microscope revealed a reduction in the viral load greater than 99%, and in particular of 2.44 log, after a contact time of 60 seconds, so that the CMV910 formulation also proves to be very effective against the EHV1 virus.

### Example 4

For this test, a formulation CMV910 was prepared as described in example 3.2, and it was mixed with a percentage of 97% in a solution comprising cells coming from an African green monkey renal epithelial cell line (Vero-E6) infected by the SARS-CoV-2 virus, with a viral load of 6.10⁷ TCID50/ml. The exposure time of the formulation to the virus was 60 seconds.

At the end of this contact, a reduction in the viral load greater than 99.99% was observed, which demonstrates an increased and rapid virucidal efficacy of the CMV910 formulation against the SARS-CoV-2 virus.

### Example 5

For this series of tests, formulations CMV40, CMV42, CMV43 and CMV44 identical to those used in example 3 were prepared, as well as a fifth formulation CMV45 according to the invention, the composition of which differs from that of the CMV42 formulation by the addition of 2% by weight of p-menthanediol as moisturising compound.

These five formulations were each mixed, at a percentage of 97%, in a solution comprising cells coming from an embryonic cell line coming from a human kidney (HEK) infected by an ADV5 adenovirus carrying a fluorescent protein. For each test sample, the exposure time of the formulation to the virus was 60 seconds.

Next, a well was formed for each sample obtained on an examination slide, in order to be able to observe under the microscope the number of infected cells per well after this time of 60 seconds, compared with control wells containing only infected cells without antimicrobial formulation.

Microscope observation revealed, after 60 seconds of contact with the ADV5 virus:
for the “reference” formulation CMV40 not containing organic acid, a zero reduction in viral load;
for the “test” formulations CMV43 and CMV45, a reduction in the viral load of the order of respectively 34% and 35%; and
for the “test” formulations CMV42 and CMV44, a reduction in the viral load of close to 70%, i.e. respectively 69% and 68%.

Thus, this series of tests revealed an increased and rapid virucidal efficacy against the ADV5 virus of formulations comprising the following combination:
as antimicrobial organic acid:
   2% by weight of lactic acid;
as booster compounds:
   6.5% by weight of glyceryl stearate (amphiphilic);
   3% by weight of glyceryl laurate (amphiphilic);
   0.4% by weight of xanthan (amphiphilic);
as bases:
   0.05% by weight of a clay;
   sodium hydroxide, at a sufficient level to confer on the formulation a pH of 5;
as dermocosmetic compounds:
   2% by weight of almond oil (hydrating);
   0.1% by weight of shea butter (hydrating);
   2% by weight of glycerol (moisturising);
0.05% by weight of microcrystalline cellulose;
1.4% by weight of hexanediol-based solvent;
the remainder in water.

### Example 6.1

For this series of tests, a “reference” formulation CMVT and four “test” formulations CMV2, CMV3, CMV4 and CMV5 were prepared according to the invention, which all contained:
5% by weight of glyceryl stearate;
0.5% by weight of xanthan;
5% by weight of almond oil;
4.5% by weight of glycerol;
a sodium hydroxide base at a level adapted for conferring on said formulation a pH of 5;
the remainder in water.

If the “reference” formulation CMVT contained only the aforementioned compounds, and no organic acid, the following compounds were added to each test formulation:
in common:
   2% by weight of lauric acid;
   1% by weight of oleic acid;
   2% by weight of glyceryl laurate;
as specific ingredients:
   for the CMV3 formulation:
      2% by weight of p-menthanediol; or
   for the formulations CMV2, CMV4, CMV5:
      2% by weight of lactic acid; in particular with
         0.08% by weight of cetrimonium chloride (CMV5); or
         1% by weight of methyl cyclodextrin (CMV4).

With each of these five formulations, two cloths of the Textile StrerilWip type were impregnated on their surface with a quantity of 60 mg/2.25 cm², with a pause time of respectively 3 minutes and 3 hours for each of these two cloths.

Next, these preimpregnated cloths were coated with a solution containing Vero-E6 cells infected by the SARS-CoV-2 virus, with a viral load of 10 μl at 10⁵ TCID50/2.25 cm², counting a formulation/virus contact time of 5 minutes.

At the end of this contact time of 5 minutes, the cloths were observed, and there were found:
for the cloth treated with the “reference” formulation CMVT, a zero reduction in the viral load, both after 3 minutes and after 3 hours of impregnation;
for the cloths treated with the “test” formulations, a reduction in the viral load greater than 81.3% after 3 minutes of impregnation, and greater than 96.4% after 3 hours of impregnation.

Thus, this series of tests allowed to show the efficacy against the SARS-CoV-2 virus of a textile treatment with a formulation comprising at least the following ingredients:
as antimicrobial organic acids:
   2% by weight of lauric acid;
   1% by weight of oleic acid;
as base:
   sodium hydroxide at a level adapted for conferring on said formulation a pH of 5;
as booster compounds:
   5% by weight of glyceryl stearate;
   2% by weight of glyceryl laurate;
   0.5% by weight of xanthan;
as additional dermocosmetic ingredients:
   5% by weight of almond oil;
   4.5% by weight of glycerol;
the remainder in water.

### Example 6.2

For this series of tests, a “test” formulation CMV910 was prepared as described in examples 3.2 and 4, which was applied to a Textile StrerilWip cloth with an application time of 3 hours and with a quantity of 60 mg/2.25 cm².

A solution of infected cells identical to that used in example 6.1 was next applied to this textile, counting an application time of 5 minutes, at the end of which we observed the results, which disclosed a reduction in the viral load greater than 98% after 3 hours of impregnation of the formulation tested, i.e. a result slightly superior to that observed with the “test” formulations of example 6.1.

1. A topical antimicrobial formulation comprising an acidic substance comprising at least one cosmetically acceptable organic acid diluted in an aqueous solvent while having a deleterious action against microbes, said acidic substance having an acidity constant pKa that is less than 6, wherein said formulation comprises:
between 1% and 10% by weight of the acidic substance;
a basic substance comprising at least one base, the basic substance being present in the formulation in an amount such that the pH of said formulation is between the pKa of the acidic substance and 8; and
between 0.1% and 15% by weight of a booster substance that is capable of biochemically interacting with a membrane of the microbes to facilitate the deleterious action of the acidic substance on said microbes, said booster substance comprising at least one cosmetically acceptable compound selected from amphiphilic compounds, chelating compounds having an ability to associate with divalent ions or sterols, and polyphenolic compounds.

2. The topical antimicrobial formulation according to claim 1, wherein the acidic substance comprises at least one organic acid selected from the group consisting of lactic acid, citric acid, tartaric acid, acetic acid, adipic acid, anisic acid, malic acid, succinic acid, benzoic acid, cholic acid, ascorbic acid, dehydroacetic acid, deoxycholic acid, glucoronic acid, glycolic acid, fumaric acid, formic acid, gluconic acid, glutamic acid, guanylic acid, folic acid, fulvic acid, sorbic acid, glycyrrhizic acid, kojic acid, itaconic acid, mellisic acid, hyaluronic acid, amino acids, glutaric acid, glyoxylic acid, humic acids, hippuric acid, inosic acid, levulinic acid, perillic acid, quinic acid, tauric acid, ribonic acid, salicylic acid, shikimic acid, trenaxamic acid, thujic acid, tiglic acid, ursolic acid, usnic acid, vanillic acid, and valproic acid.

3. The topical antimicrobial formulation according to claim 1, wherein the basic substance comprises at least one base selected from the group consisting of organic acid salts, clays, sodium hydroxide, potassium hydroxide, triethanolamine, butylethanolamine, disodium carbonates, dipotassium carbonates, disodium phosphates, and dipotassium phosphates.

4. The topical antimicrobial formulation according to claim 1, wherein the basic substance is present in an amount such that said formulation has a pH of about 5.

5. The topical antimicrobial formulation according to claim 1, wherein the booster substance comprises at least one amphiphilic compound having a critical micelle concentration (CMC) value of between 1 μM and 400 mM.

6. The topical antimicrobial formulation according to claim 5, wherein the booster substance comprises at least one amphiphilic compound selected from the group consisting of glyceryl laurate, glyceryl caprate, glyceryl caprylate, glyceryl palmitate, glyceryl stearate, quaternary ammoniums, and saponins.

7. The topical antimicrobial formulation according to claim 5, wherein the booster substance comprises at least one cosmetically acceptable amphiphilic compound selected from the group consisting of from fatty alcohols, ethoxylated alcohols, an amphiphilic compound with betaine, phosphate or sulfate group, alkylated sugars, polyglyceryl, and polysorbate.

8. The topical antimicrobial formulation according to claim 1, wherein the booster substance comprises at least one chelating compound that has a LogK constant of interaction with divalent ions or sterols greater than 4.

9. The topical antimicrobial formulation according to claim 8, wherein the booster substance comprises at least one chelating compound selected from the group consisting of EDTA (ethylenediaminetetraacetic acid), cyclodextrin, phytic acid, oxalic acid, salts thereof, and derivatives thereof.

10. The topical antimicrobial formulation according to claim 1 wherein the booster substance comprises at least one polyphenolic compound selected from the group consisting of quercetin, oleuropein, cafeic acid, gallic acid, epigallocatechin, epigallocatechin-gallate, tannins, tannic acids, fulvic acids, humic acids, plant extracts, in particular based on olive, tea, cocoa bean, coffee bark, and cassia bark.

11. The topical antimicrobial formulation according to claim 1, wherein the formulation furthermore comprises at least one additional compound having a dermocompatible function.

12. The topical antimicrobial formulation according to claim 11, wherein the formulation comprises at least one additional compound that has at least one dermocosmetic function selected from hydrating functions, emollient functions, moisturising functions and/or relipidising functions.

13. The topical antimicrobial formulation according to claim 12, wherein the formulation comprises at least one dermocosmetic compound selected from the group consisting of almond oil, shea butter, glycerol, triheptanoin, p-menthanediol, dibutyl adipate, and a fatty alcohol.

14. The topical antimicrobial formulation according to claim 1, wherein the formulation furthermore comprises an additional component having a homogenising function.

15. The topical antimicrobial formulation according to claim 14, wherein the formulation comprises an additional organic solvent.

16. The topical formulation of claim 14, wherein the additional component having the homogenising function is microcrystalline cellulose.

17. The topical formulation of claim 5, wherein the booster substance comprises (i) between 5% and 7% by weight of glyceryl stearate; (ii) between 0.5% and 3.5% by weight of glyceryl laurate; and (iii) between 0.2% and 0.7% by weight of xanthan.

18. The topical formulation of claim 13, wherein the dermocosmetic compound comprises between 1% and 5% by weight of glycerol and between 1% and 6% by weight of almond oil.
